(19) United States
(12) Patent Application Publication
(10) Pub. No.: US 2025/0009988 A1
(43) Pub. Date: Jan. 9, 2025

(54) INJECTION DEVICE

(71) Applicant: Allergan, Inc., Irvine, CA (US)

(72) Inventors: Ahmet Tezel, Fort Worth, TX (US); Kevin McNERNEY, Huntington Beach, CA (US); Christopher S. MUDD, Lake Forest, CA (US); Blake R. STORIE, Laguna Niguel, CA (US); Bastien MANDAROUX, Cran Gevrier (FR)

(21) Appl. No.: 18/646,562

(22) Filed: Apr. 25, 2024

Related U.S. Application Data
(63) Continuation of application No. 16/358,553, filed on Mar. 19, 2019, now Pat. No. 11,992,668.
Application No. 16/358,553 is a continuation of application No. 12/629,480, filed on Dec. 2, 2009, now Pat. No. 10,232,129.
(60) Provisional application No. 61/139,430, filed on Dec. 19, 2008.
(60) Provisional application No. 61/119,298, filed on Dec. 2, 2008.

(51) Int. Cl. A61M 5/34; A61M 5/32

(52) U.S. Cl. CPC A61M 5/347; A61M 5/3293; A61M 2210/04

(57) ABSTRACT

A needle assembly for an injection device is provided to facilitate delivery of a dermal filler. The assembly includes a cannula and a hub. The hub can be coupled to a syringe. The hub includes an inner bore that has a proximal retention section, a stepped section, and a cannula retention section. The stepped section, the proximal retention section, and the cannula retention section are configured to reduce the dead space within the inner bore for preventing detachment of the hub from the syringe during an injection procedure.

### RELATED APPLICATIONS

This application is a continuation of U.S. patent application Ser. No. 12/629,480, filed on Dec. 2, 2009, which claims the benefit of U.S. Provisional Patent Application No. 61/119,298, filed on Dec. 2, 2008 and U.S. Provisional Patent Application No. 61/139,430, filed on Dec. 19, 2008, the entire disclosure of each of which is incorporated herein by this specific reference.

### BACKGROUND

The present invention is generally directed to non-surgical devices for the correction of skin contour defects and aging and is more particularly directed to an injection device for enabling introduction of a dermal filler into peripheral locations of a human body through a cannula.

Dermal fillers such as, such as Juvederm™, a hyaluronic acid based formulation, have been used for the treatment of nasal labial folds, lip augmentation, tear troughs, and for facial volumizing through the use of very fine syringe needles.

The dermal fillers are of high viscosity and this provides for effective, and preferably substantially uniform, suspension of the dermal filler into a peripheral location.

A relatively small needle size, gauge, is preferred for delivery of the dermal filler in order to lessen complications and recovery time. However, in combination with the relatively high viscosity of the dermal filler, a problem can arise with regard to needle assembly separation from the syringe due to the high pressure generated by a piston reacting on the high viscosity dermal filler in order to eject the filler from the syringe through a fine needle and into the patient.

The present invention overcomes this problem by providing an ejection device which eliminates, or substantially reduces, the probability of needle assembly/syringe separation during a procedure.

### SUMMARY OF THE INVENTION

An injection device in accordance with the present invention generally includes a syringe having a body with a piston disposed therein and an open distal end.

A needle assembly is provided which includes a cannula and a luer connector engagable with the syringe distal end. The needle or cannula, hereinafter “cannula”, has a gauge of about 18 to a gauge of about 25 or greater. The luer connector includes a hub with a distal end supporting the cannula and a proximal end matable with the syringe distal end.

In one embodiment, the present invention further includes a viscous fluid, for example, a hyaluronic acid-based dermal filler, disposed in the syringe's body and which is injectable by the piston into a peripheral location of a human or animal body through the cannula.

The mating engagement, for example, between the hub and the syringe distal end is provided by internal threads disposed in the syringe distal end and external threads disposed on the hub, as well as between a tapered syringe cone and a tapered inside surface of the hub. The internal threads have a pitch which is sufficient to prevent detachment of the hub from syringe distal end during injection of the viscous fluid into a peripheral location of a human or animal body.

More particularly, in one embodiment, the internal threads have a pitch of between about 2 mm and about 5 mm. For example, the internal threads have a pitch of about 3 mm. The internal thread may be double lead screw threads. In addition, the external threads disposed on the hub further may also be double lead screw threads and the double lead screw threads provide an advantage of enabling the hub to travel twice the distance into mating engagement with the syringe distal end with each single turn of the hub. In other embodiments, the internal threads have a pitch of about 2.0 mm, about 3.0 mm, about 3.5 mm, about 4 mm, about 4.5 mm or about 5 mm. In addition, the external threads may have a pitch of about 2.0 mm, about 3.0 mm, about 3.5 mm, about 4 mm, about 4.5 mm or about 5 mm.

In a specific embodiment, the external threads comprise single circumference double lead screw threads as will be hereinafter described in greater detail.

In addition, a cavity, for example, a stepped cavity, is disposed in the hub. The cavity serves not only reduces dead space in the syringe but also significantly reduces the possibility of detachment of the hub from the syringe distal end during operation of the piston to eject the viscous fluid through the cannula. Thus, the cavity can be considered a hub retention cavity.

### BRIEF DESCRIPTION OF THE DRAWINGS

The advantages and features of the present invention will be better understood by the following description when considered in conjunction with the accompanying drawings in which:

FIG. 1 is an illustration of use of an injection device in accordance with the present invention for injecting a viscous fluid into a peripheral location of the human, generally showing a syringe having a body with a piston and a needle assembly;

FIG. 2 is a cross sectional view of the syringe shown in FIG. 1 illustrating more clearly showing the syringe body along with a needle assembly with a luer connector engagable with a distal end of the syringe along with a viscous fluid disposed in the syringe body;

FIG. 3 is an enlarged cross sectional view of the mating engagement between a luer connector hub and a distal end of the syringe specifically illustrating internal threads disposed in the syringe distal end and external threads along the hub for enabling the mating engagement, along with a cavity disposed in the hub configured as a hub retention cavity.

FIG. 4 is end view of the hub illustrating double lead screws;

FIG. 5 is an enlarged cross section view of the needle assembly and an open distal end of the syringe body showing a tapered syringe cone engageable with a tapered inside surface of the needle assembly hub; and

FIG. 6 is a cross-section view of the needle assembly engaged with the syringe's open distal end.

### DETAILED DESCRIPTION

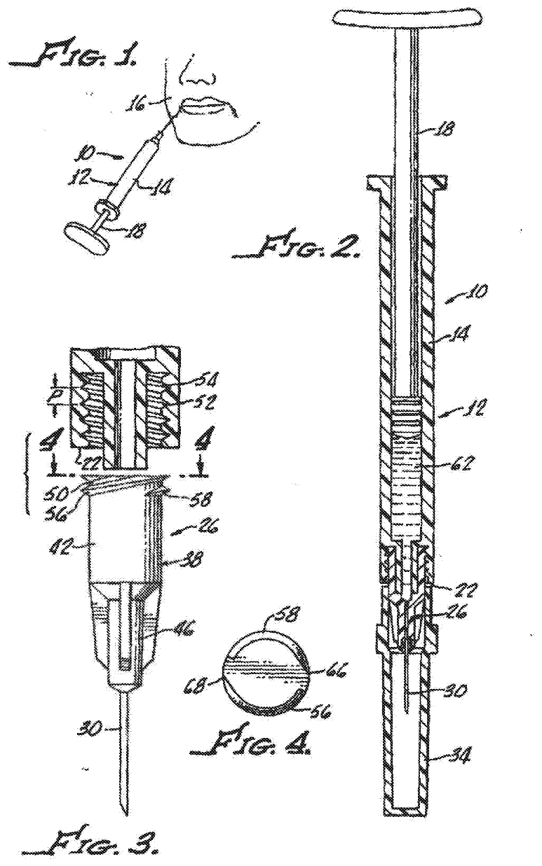

With reference to FIG. 1, there is shown an injection device 10 in accordance with the present invention having a syringe 12 with a body 14 with a piston 18 disposed therein.

With additional reference to FIGS. 2 and 3, the syringe 14 includes an open distal end 22, and a needle assembly 26. The needle assembly includes a cannula 30 along with a sheath 34 and a luer connector 38 engagable with the syringe open distal end 22, which includes a hub 42 having a distal end 46 and a proximal end 50 matable with the syringe distal end 22.

Internal threads 52, 54 and external threads 56, 58 enable the mating engagement. Pitch (P) of the threads 52, 54 enables the ejection of viscous fluid 62 through the cannula 30 upon operation of the piston 18 without separation of the hub 42 from the syringe open distal end 22 during ejection of the viscous fluid 62 as illustrated in FIG. 1.

In some embodiments, the viscous fluid 62 is a dermal filler. In some embodiments, the viscous fluid is a hyaluronic acid-based fluid having a viscosity between about 50,000 cps and about 500,000 cps, measured at about 25° C. with a controlled stress rheometer (RS600 TA Instrument) and a cone plate geometry (40 mm, 2°). In some embodiments, the viscous fluid is a hyaluronic acid based dermal filler having a viscosity greater than about 130,000 cps.

When measured with a traction column test (Versatest, Mecmesin), at 13 mm/min (extrusion speed) and a needle with a gauge between 21 G to 32 G, the viscous fluid may have an extrusion force of between about 5 N up to about 200 N, more specifically, an extrusion force of between about 10 N to about 150 N.

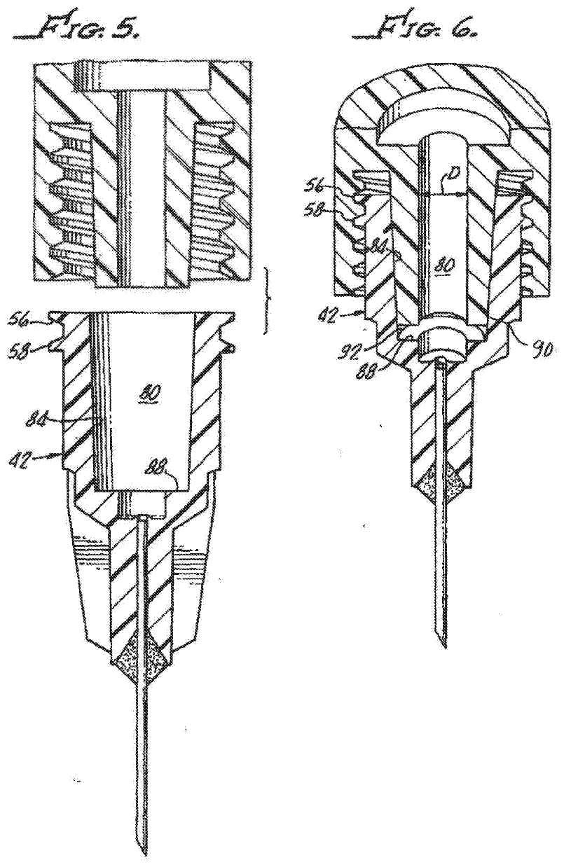

In an exemplary embodiment, the pitch of the hub threads 52, 54 is between about 2 mm and about 5 mm. Preferably, the pitch is about 3 mm. The threads 52, 54 and 56, 58 are preferably at least double lead screw threads, although triple, and even quadruple lead threads may used. As most clearly shown in FIG. 4, in one particular embodiment, the threads 52, 54 have two thread starts 66, 68 at a 180° radial displacement from one another.

As hereinabove noted, this enables rapid engagement of the hub 42 with the syringe open distal end 22.

In one embodiment, the hub 42 is formed from a low elasticity material, for example, an acrylic or a polycarbonate, rather than polypropylene typically used for syringe hubs, as hereinafter described in connection with needle retention tests. This further enhances the prevention of detachment of the hub 46 from the syringe open distal end 22.

With reference to FIGS. 5 and 6, hub 42 includes a hub retention cavity 80 defined by an inside surface 84 of the hub 42 and a step 88, thus providing a unique stepped interior surface. When torqued to the syringe and maximally seated as shown in FIG. 6, a tapered syringe cone 90 stops short of the step 88 and creates a dead space 92 which is reduced by the step 88. Engagement between the cone 90 and inside surface 84, which is also tapered, provides a seal therebetween. Low dead space reduces the amount of fluid that cannot be administered. This is important in that the fluids to be administered, by injection, often are very expensive.

The step 88 unexpectedly prevents or substantially reduces the possibility of detachment of the hub 42 from the syringe cone 90 and syringe open distal end 22 during injection of the viscous fluid 62.

That is, the average detachment force is significantly greater with the step 88 formed in the cavity hub retention cavity 80 of the hub 42 as was discovered during comparison study as follows:

Test methods used in this study utilized equipment designed by Omnica Corporation specifically for torque setting and detachment testing.

The torque set unit utilizes a numerically controlled motor and torque sensor which tightens the needle hub 42 onto the syringe open distal end 22 and a pre-determined torque setting testing is shown that the static friction between the needle hub 42 and the syringe open distal end 22 materials causes more overshoot than observed with standard polypropylene hubs (not shown).

A detachment tester utilizes a numerically controlled motor driving linear carriage against a forced transducer.

In a comparison test, all of the hubs (not shown) were attached to the syringe open distal end 22 and cone 90 with a torque of 0.07 Nm (Newton meter). All of the tests were performed on 0.8 cc syringes having identical dimensions, for example, syringe open distal end 22 having an entry diameter D (see FIG. 6), of about 4 mm.

The results are shown in Table 1 for nominal torque static test needle retention with various design attribute combinations.

Table 1 shows that the conventional polypropylene hub mated to a syringe with 5 mm threads has an average detachment force 46.1 N (Newton) when the hub is attached to the syringe with a force of 0.07 Nm.

TABLE 1
Nominal torque Static Test Needle Retention
with Various Design Attribute Combinations

| | Average Detachment |
|---|---|
| Needle Design | Force (N)¹ @ 0.07 Nm |
| Conventional Polypropylene Hub, Syringe | 46.1 |
| with 5 mm Threads | |
| Conventional Polypropylene Hub, Syringe | 56.2 |
| with 3 mm Threads | |
| Polycarbonate Hub and Syringe with 5 mm | 83.2 |
| Threads | |
| Polycarbonate Hub and Syringe with 3 mm | 96.0 |
| Threads | |
| Polycarbonate Hub and Syringe with 3 mm | 200+ |
| Threads and Stepped Cavity | |

Slight improvement shown in the conventional polypropylene hub attached to the syringe body with 3 mm threads, and an average detachment force of about 56.2 N.

Utilizing polycarbonate instead of polypropylene for the hub 42 results in an average detachment force of 83.2 N with 5 mm threads utilized for attachment.

Combination of polycarbonate hub 42 with 3 mm threads without a step 88 results in a 96 N average detachment force.

Surprisingly, the detachment force for the hub 42 with 3 mm threads incorporating the dead space reducing step 88 results in an average detachment force of more than 200 N.

It is contemplated that other structures may be useful in reducing dead space in a manner effective to increase detachment force relative to conventional syringe/needle combinations. For example, it is contemplated that reduced dead space can be accomplished, within the scope of the present invention, by reducing an outer diameter of the syringe luer tip, increasing an inner diameter of the hub cavity, and/or other ways effective to increase the engagement length of the syringe tip and the hub.

### EXAMPLE

A multi-center, double-blind, randomized parallel-controlled prospective comparison study was undertaken to test needle disengagement rates. This study tested needle disengagement rates of a Polycarbonate Hub and Syringe with 3 mm Threads and Stepped Cavity in accordance with the present invention (study device) in comparison to a conventional polypropylene hub and syringe with 5 mm threads (conventional device). Needles of each of the conventional devices and the study devices tested were all 30 G X½″ needles. The material injected for the study was Juvederm™ Ultra Injectable Gel (dermal filler).

There were up to 288 study participants. Each study participant underwent treatment for improving his or her bilateral nasolabial folds using 2 syringes of dermal filler packaged in either the conventional device or the study device.

The results demonstrated a significant difference in needle disengagement rates between the conventional device and the study device. No disengagements were reported for the study device. Between 3% and 7% of the needles of the conventional device unintentionally disengaged during injection.

Although there has been hereinabove described a specific injection device in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1-20. (canceled)

21. An injection device for delivering a dermal filler, the injection device comprising:
a syringe having a body with a piston disposed therein and an open distal end;
a needle assembly comprising a cannula and a luer connector engageable with the syringe distal end, the luer connector comprising a hub with a distal end supporting the cannula and a proximal end mateable with the syringe distal end;
a hyaluronic acid-based dermal filler having a viscosity between about 50,000 cps and about 500,000 centipoise, measured at about 25° C. with a controlled stress rheometer (RS600) and a cone plate geometry (40 mm, 2°), disposed in the body and injectable by the piston into a peripheral location of a human or animal body through the cannula;
a hub retention cavity, disposed in the hub, for preventing detachment of the hub from the syringe distal end during injection of the dermal filler, the hub retention cavity being defined by an inside surface of the hub and a step; and
internal threads disposed in the syringe distal end and external threads disposed on the hub enabling mating engagement and ejection of the viscous fluid through the cannula upon operation of the piston.

22. The device according to claim 21, wherein the syringe includes a tapered syringe cone and the inside surface of the hub is correspondingly tapered to the tapered syringe cone, to thereby provide a seal therebetween.

23. The device according to claim 22, wherein, when the hub is torqued to the syringe and maximally seated, the tapered syringe cone stops short of the step and creates a dead space which is reduced by the step.

24. The device according to claim 21, wherein the hub is formed from polycarbonate.

25. The device according to claim 21, wherein the internal threads comprise double lead screw threads.

26. The device according to claim 21, wherein the external threads comprise double lead screw threads.

27. The device according to claim 26, wherein the external threads comprise single circumference double lead screw threads.

28. The device according to claim 21, wherein the hyaluronic acid-based dermal filler has a viscosity of at least about 130,000 cps.
